(19) United States
(12) Patent Application Publication
(10) Pub. No.: US 2015/0230726 A1
(43) Pub. Date: Aug. 20, 2015

(54) COMPREHENSIVE HEALTH ASSESSMENT TOOL FOR IDENTIFYING ACQUIRED ERRORS OF METABOLISM

(71) Applicant: Lynn S. Greaves, Kansas City, MO (US)

(72) Inventor: Lynn S. Greaves, Kansas City, MO (US)

(73) Assignee: Enzymes, Inc., Kansas City, MO (US)

(21) Appl. No.: 14/277,592

(22) Filed: May 14, 2014

Related U.S. Application Data
(60) Provisional application No. 61/823,038, filed on May 14, 2013.

(51) Int. Cl. A61B 5/053

(52) U.S. Cl. CPC A61B 5/0531; A61B 5/0537

(57) ABSTRACT

A method of comprehensive health assessment includes using a biocommunication or bioenergetic device to measure signals sent across or through the body. Fluctuations in galvanic skin response are measured and transmitted to a computer or computing device and compared to a library of possible stimulus sources, each associated with a predetermined electrical signature.

### CROSS REFERENCE TO RELATED APPLICATION

This application claims the benefit of the prior filed, co-pending provisional application, Ser. No. 61/823,038, filed May 14, 2013.

### BACKGROUND OF THE INVENTION

1. Field of the Invention

This invention relates generally to the use of health assessment tools to evaluate the occurrence of acquired errors of metabolism. Results from these evaluations are the basis for treatment protocols to resolve acute or chronic metabolic stress patterns, thereby preventing the progression of symptoms to more serious disease states.

2. Description of the Related Art

The metabolic pathways of healthy living organisms have been thoroughly elucidated by the scientific community and full descriptions can be found in most, if not all, biochemistry textbooks. It is well-established that each metabolic pathway is interconnected with one or more other pathways and requires an adequate supply of specific substrates, enzymes, co-factors and biochemical energy in order to function properly. Research has further shown that as an individual passes through its life cycle, its metabolic pathways are constantly changing in response to stress from external sources such as pathogens, allergens, nutritional deficiencies, energetic depletion, physical and emotional trauma, and accumulation of toxins from the environment (toxic overload). If the stress is continuous, the metabolic network becomes inefficient due to impairment of biochemical pathways and compensatory biochemical rerouting through different pathways.

Each pathway in the metabolic network affects one or more other pathways. If a pathway is functioning too slowly, it will slow down the production line which will back up into pathways that flow into it; and if a pathway is functioning too fast, it will produce too much substrate for the pathways downstream from it to handle. A metabolic pathway can be functioning too slow as the result of inhibited enzymes, inadequate supply of cofactors or a backlog from a downstream pathway not performing efficiently. A pathway can be functioning too fast as a result of enzyme up-regulation and an overproduction of biochemical substrates in an upstream pathway or over-supplementation of cofactors.

Stress patterns, commonly known as acquired errors of metabolism, begin to manifest as symptoms when the body reaches a critical threshold of impairment. This phenomenon becomes most evident in areas of the body that tend to be genetically weak in an individual. Many common signs and symptoms of chronic health disorders are directly attributable to acquired errors of metabolism. In many cases, the dysfunctional pattern has become the “norm” with the body no longer attempting to correct the errors but simply adapting to them.

Medical care continues to diagnose and treat chronic diseases based on symptomatology, confirmed by the results of standard laboratory analyses and diagnostic testing. Knowledge of metabolic pathways has allowed the pharmaceutical industry to create drugs that will target specific pathways, or parts of pathways, as a means of alleviating common symptoms with the implication that the disease is thereby under control when the symptoms are gone. Unfortunately, this approach to medicine ascribed to by most medical doctors is reactive, not proactive, and frequently does not resolve the acquired metabolic error causing the symptoms. Therefore, the disease is not truly under control as the metabolic imbalance will manifest itself as the same set of symptoms, or a completely new set of symptoms, in the future.

For example, the mechanism of action for aspirin involves irreversible inhibition of the enzyme cyclooxygenase, suppressing the production of prostaglandins and thromboxanes, thereby reducing pain and inflammation. This was the intended effect of the medication but the acquired metabolic error that produced excessive inflammation in the first place is not addressed by this treatment. If the cause of the metabolic imbalance is not resolved, inflammation will continue to be a health problem for this individual throughout his life. Of possibly greater concern are the metabolic problems created by the use of drug therapy. Although pharmaceuticals generally have the intended therapeutic effect, they often have secondary undesirable effects due to the adverse impact of the drug directly on other metabolic pathways in the body or indirectly as the breakdown of the drug results in potentially toxic byproducts which the body must eliminate. Most of the Physician's Desk Reference is devoted to such secondary or side effects.

Nutritional approaches to healthcare are often more supportive of metabolic pathways than drug-oriented medicine, and can be helpful at relieving symptoms of chronic health problems. However, they still rely heavily upon symptomatology, to assess and treat an individual. Similar to drug therapy, most nutritional programs are implemented based upon the symptoms exhibited by the individual. Over the course of treatment, it becomes exceedingly difficult to determine whether any true progress is being made in correcting the underlying metabolic weakness.

Biocommunication or bioenergetics is a modality increasingly being used by those practicing nutritional therapy to obtain important information about an individual's health status. Biocommunication devices, such as those sold by Zyto Corp. and Innovative Health Technologies (Biomeridian), are known in the prior art. An example of a biocommunication device and methodologies of use are described in U.S. Pat. No. 8,099,159, which is hereby incorporated by reference.

Assessment tools of this type measure the body's energetic response to different stimuli and, if used properly, can be very effective at identifying acquired errors of metabolism. However, the various biocommunication protocols currently used by healthcare practitioners evaluate an individual during the first office visit for such a wide range of possible stressors, from allergens to emotions, that the results become difficult, if not impossible, to interpret and in some cases may be contradictory. Very few biocommunication protocols attempt to test for imbalances in metabolic pathways and those that do include one or two broad categories, such as the Krebs cycle, without the supporting detail in their libraries, or random substrates, enzymes, cofactors, coenzymes and energy sources. None of these attempts to measure metabolic imbalances provides a clear picture of which metabolic pathway is impaired. The treatment programs developed from these evaluations are often complicated and result in oversupplementation because a long list of nutritional supplements is recommended in an attempt to “clear” the entire list of stressors all at once. The cost to the individual for these types of programs is excessive and, although some improvement has been reported, the positive results are usually short-term Like drug therapy, over-supplementation can have an adverse effect on the metabolic network causing impaired function and compensatory re-routing through other pathways, both detrimental to optimal health.

### BRIEF DESCRIPTION OF THE INVENTION

The purpose of this invention is to provide a comprehensive health assessment tool to healthcare practitioners to be used in conjunction with a biocommunication or bioenergetic device to evaluate individuals for acquired errors of metabolism. The test results form the basis for treatment to resolve acute or chronic stress patterns thereby preventing the progression of imbalances to more serious disease states. The tool allows the healthcare practitioner to not only determine whether or not an imbalance exists in a particular biochemical pathway but also to pinpoint the compromised location in the pathway. The tool consists of a series of libraries containing all of the substrates, enzymes, co-factors, coenzymes and biochemical energy sources for each metabolic pathway in the body that, when programmed into the software of any biocommunication or bioenergetic device, will assist healthcare practitioners in evaluating and treating the health issues of their clients.

Other advantages of the invention will become apparent from the following description taken in connection with the accompanying figures, wherein is set forth by way of illustration and example an embodiment of the present invention.

### BRIEF DESCRIPTION OF THE DRAWINGS

FIG. 1a shows a partial listing of an exemplary comprehensive library of an embodiment of the invention programmed into a biocommunication device.

FIG. 1b is a continuation of the listing of FIG. 1a.

FIG. 1c is a continuation of the listing of FIG. 1b.

FIG. 2 shows a summary of comprehensive libraries included in an embodiment of the invention.

FIGS. 3a to 3o show a set of comprehensive libraries included in an embodiment of the invention.

FIG. 4 is a table showing nutritional and homeopathic products used for metabolic imbalances discovered using an embodiment of the invention.

### DETAILED DESCRIPTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

An embodiment of the invention comprises a health assessment tool to be used in conjunction with any commercially-available biocommunication or bioenergetic device that allows a healthcare practitioner to more thoroughly evaluate individuals for acquired errors of metabolism. The test results form the basis for treatment which has proven to be effective at resolving acute or chronic metabolic stress patterns, thereby preventing the progression of imbalances to more serious disease states. Evaluating an individual's metabolic health using a biocommunication or bioenergetic protocol that includes the invention is significantly different from any other assessment technique currently being used in healthcare and yields superior therapeutic results.

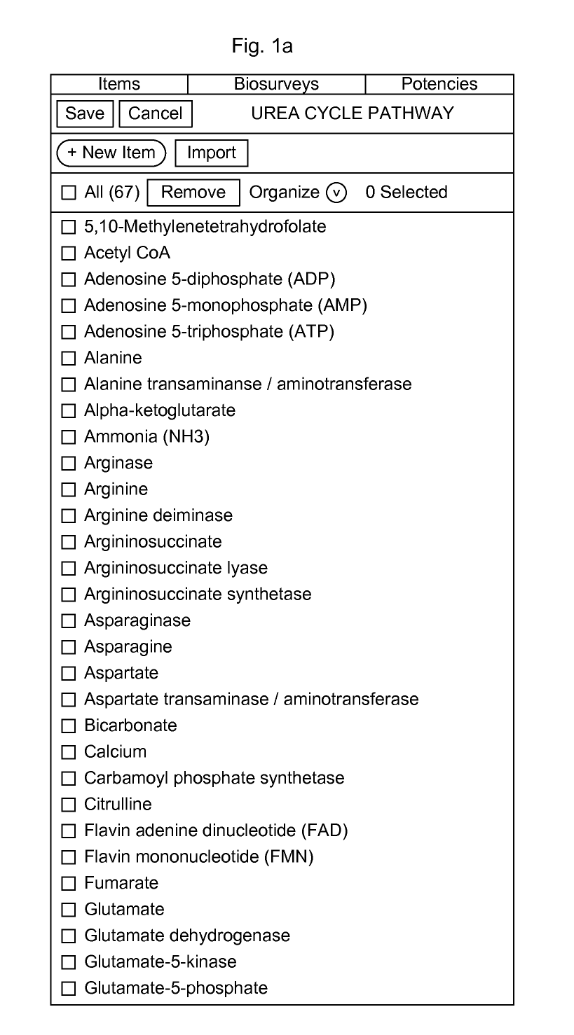

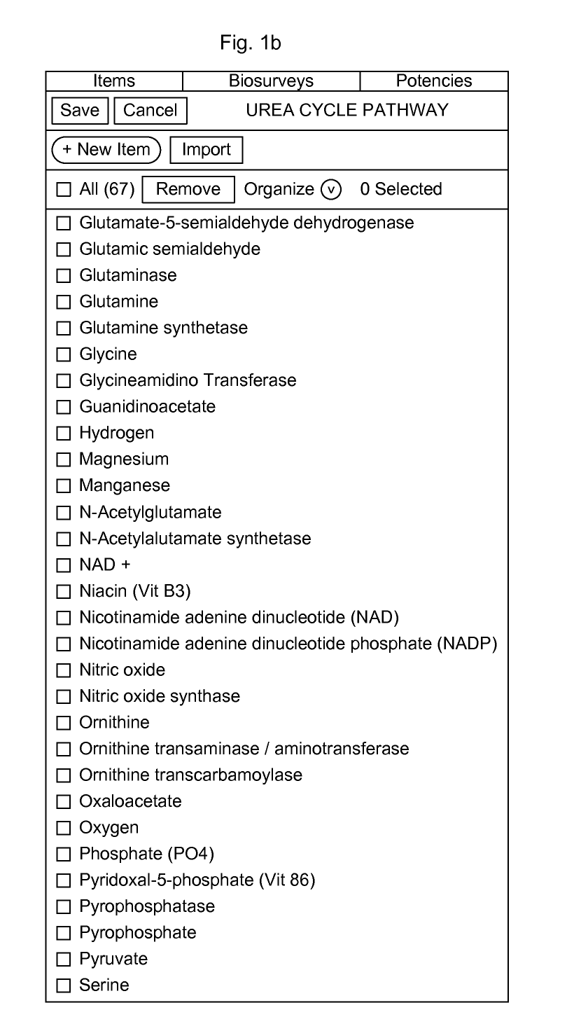

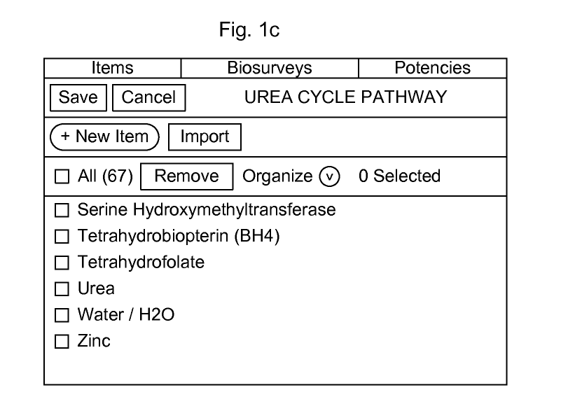

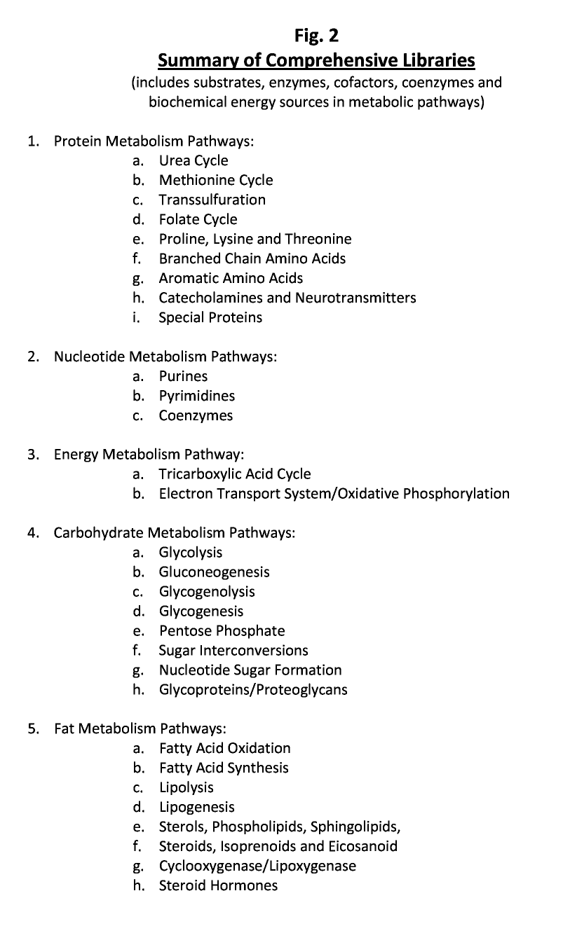

The primary component of the invention is a series of comprehensive lists or libraries containing the substrates, enzymes, co-factors, coenzymes and biochemical energy sources found in each metabolic pathway in the body, see FIG. 2, that are programmed into a biocommunication or bioenergetic device for the purpose of being incorporated into biosurveys that will measure an individual's energetic response to these items, see FIG. 1. Any permutation or alteration of the order or placement of the items in the lists or libraries programmed into the biocommunication or bioenergetic device are part of the invention. The invention encompasses all lists or libraries programmed into a biocommunication or bioenergetic device consisting of 50% or more of the substrates, enzymes, co-factors, coenzymes and biochemical energy sources scientifically recognized as being part of each metabolic pathway whether the items in the lists or libraries are designated by scientific names, common names, acronyms, abbreviations, imaginary or descriptive terms, or summary terms (i.e. Liver Detox Pathway or Urea Cycle).

Every cell in the body is networked together, constantly sending and receiving information energetically to coordinate the millions of functions in the body. This inter-cellular communication can be altered in response to stressors like viruses, allergens or pollution and when the body is no longer able to effectively handle the stressors, symptoms develop accompanied by changes in the body's energy patterns. Biocommunication or bioenergetics refers to any method that assesses the body's energetic response to pre-selected stimuli or stressors in order to obtain information about the current health status of an individual. The library of possible stimuli in the invention can be implemented on any commercially available biocommunication device capable of exchanging information between a computer and a patient's body.

The invention relies upon hololinguistic or stress technology also known as bioenergetic feedback, electro-dermal screening (EDS) or electro-acupuncture according to Voll (EAV). This technology uses a device, most commonly either a hand cradle or ohmmeter with stylus, to send the body a subtle, computer-generated impulse or digital signature representing physical stimuli such as drugs, nutrients, body functions, organs and, in this invention, metabolic pathways. A proprietary entanglement process called linking is used to associate the physical stimuli with the digital code created by the computer allowing the computer software to be able to communicate with the body. The device converts the information from digital to analog and vice versa to facilitate the communication process between the body and the computer.

Each digital signature elicits a physiological response from the body which is reflected as a change in the electrical conductivity of the skin known as a galvanic skin response (GSR). GSR technology is well-established and devices implementing this technology are generally registered with the FDA as Class II medical devices. Fluctuations in skin conductivity or GSR are measured by a biocommunication or bioenergetic device and sent back to the computer for analysis and interpretation. The data received from the device is plotted and analyzed for coherence, which is a state where two or more things exist without conflict. To determine whether there is coherence, the device begins by measuring a baseline value for the energy of the skin prior to sending stimuli to the body. Then the computer calculates deviations from the baseline or from coherence as the body responds to each digital signature.

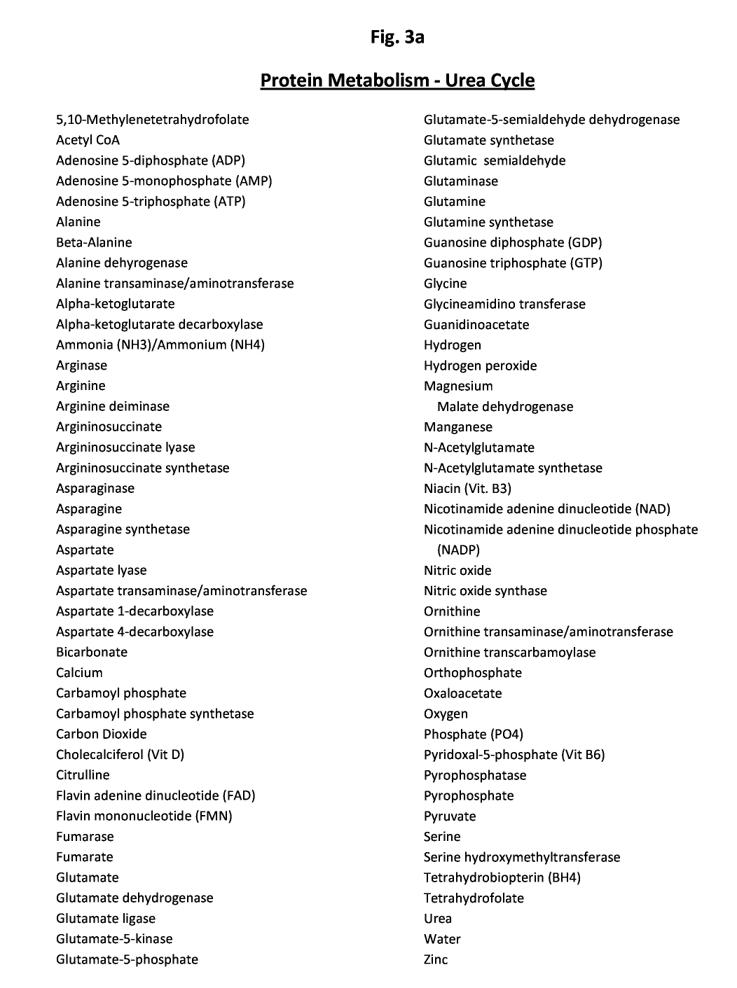

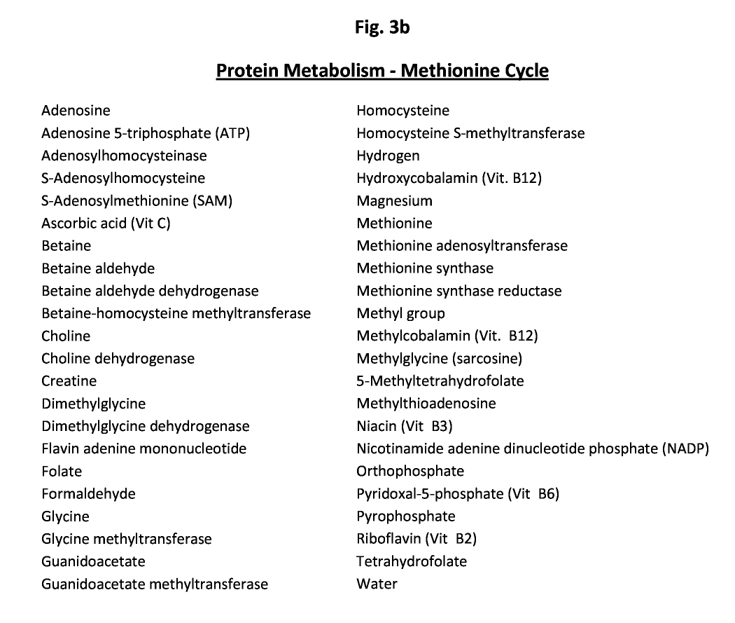

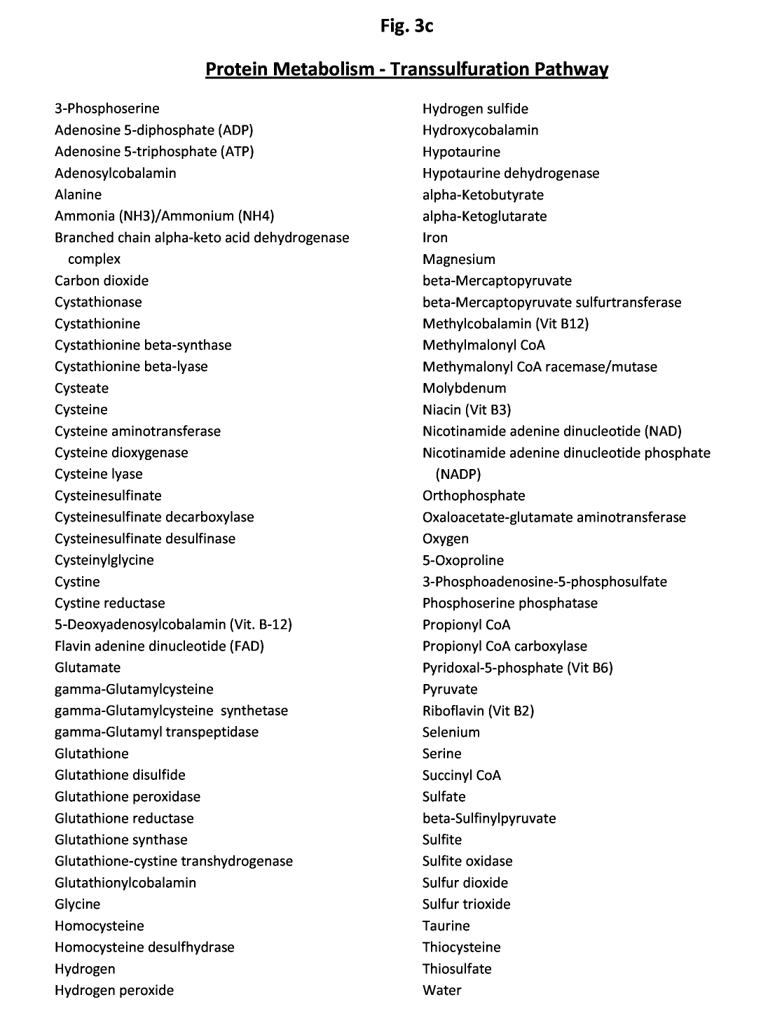

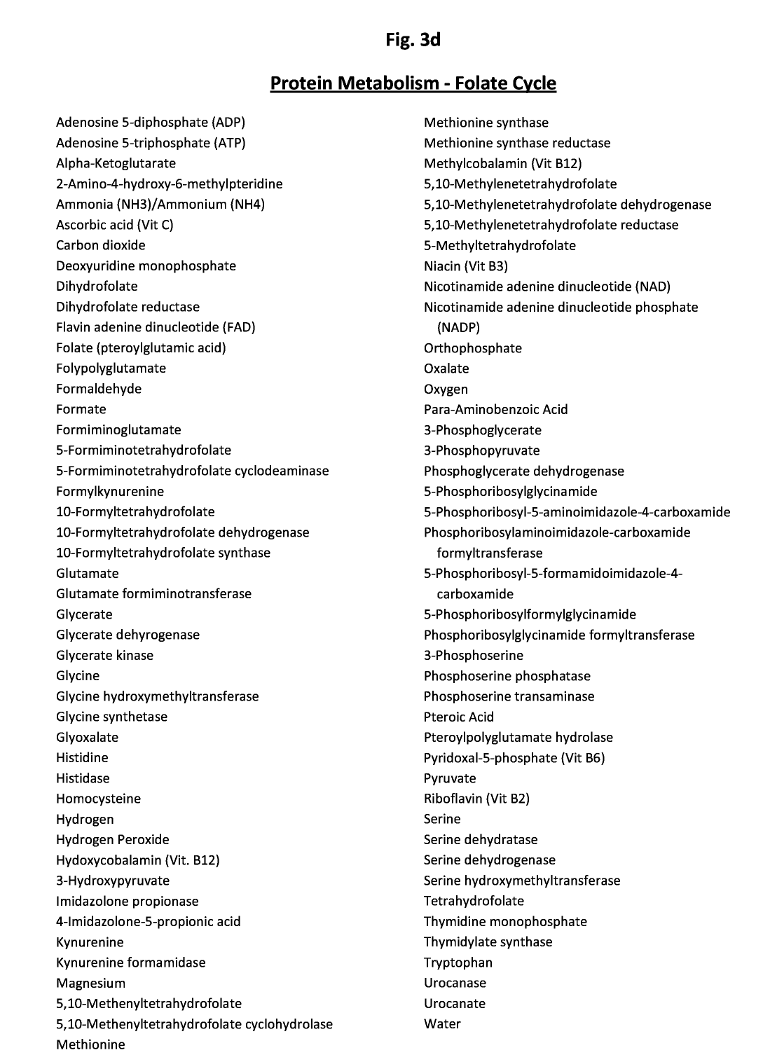

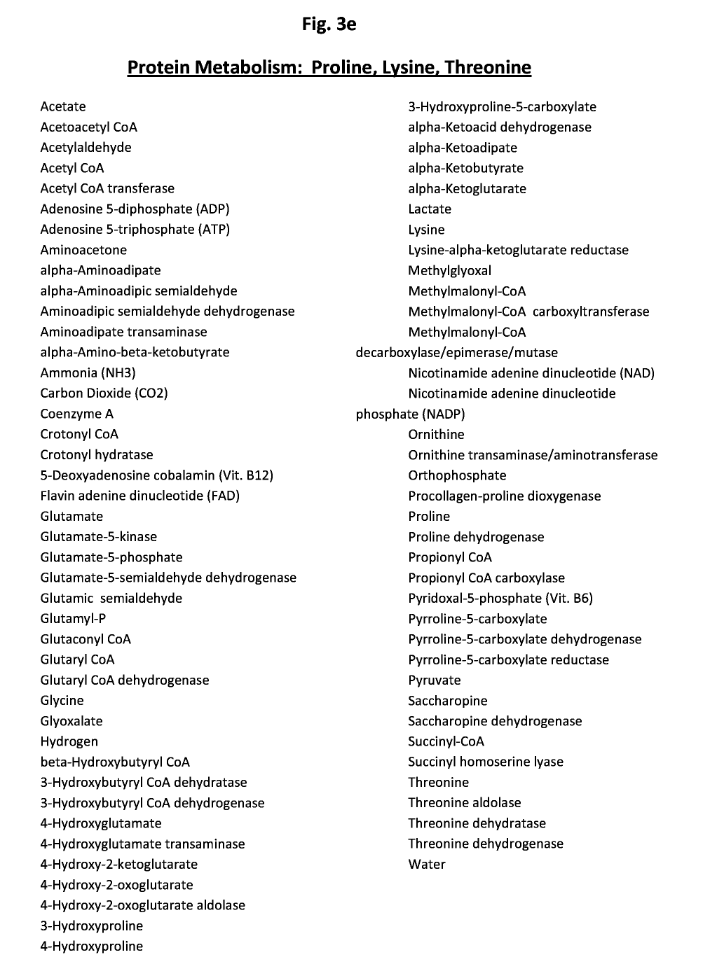

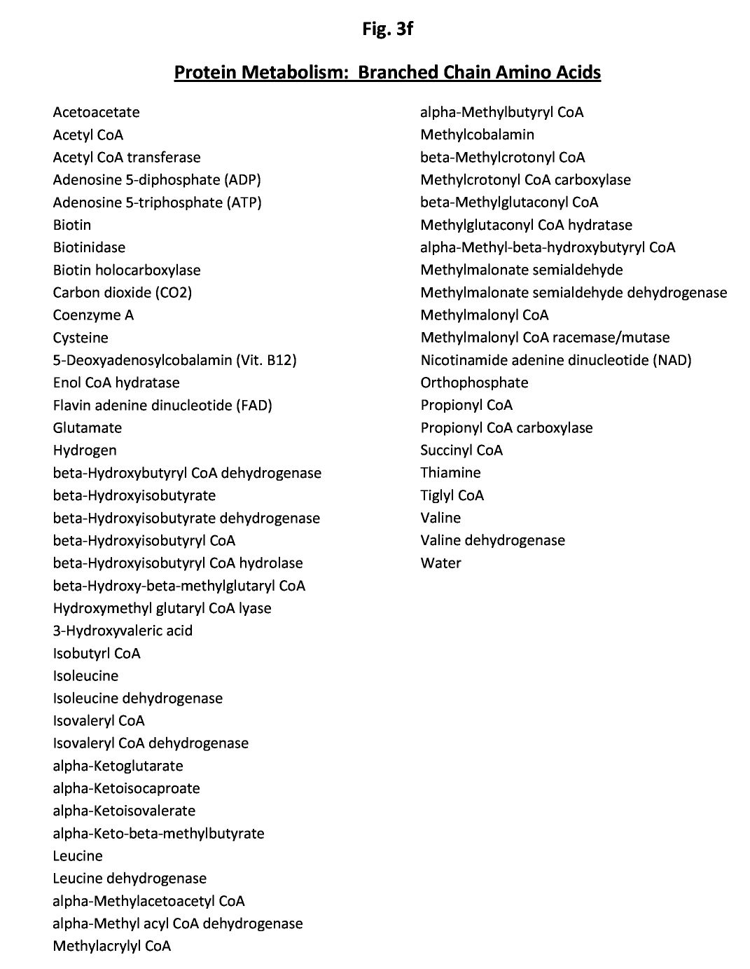

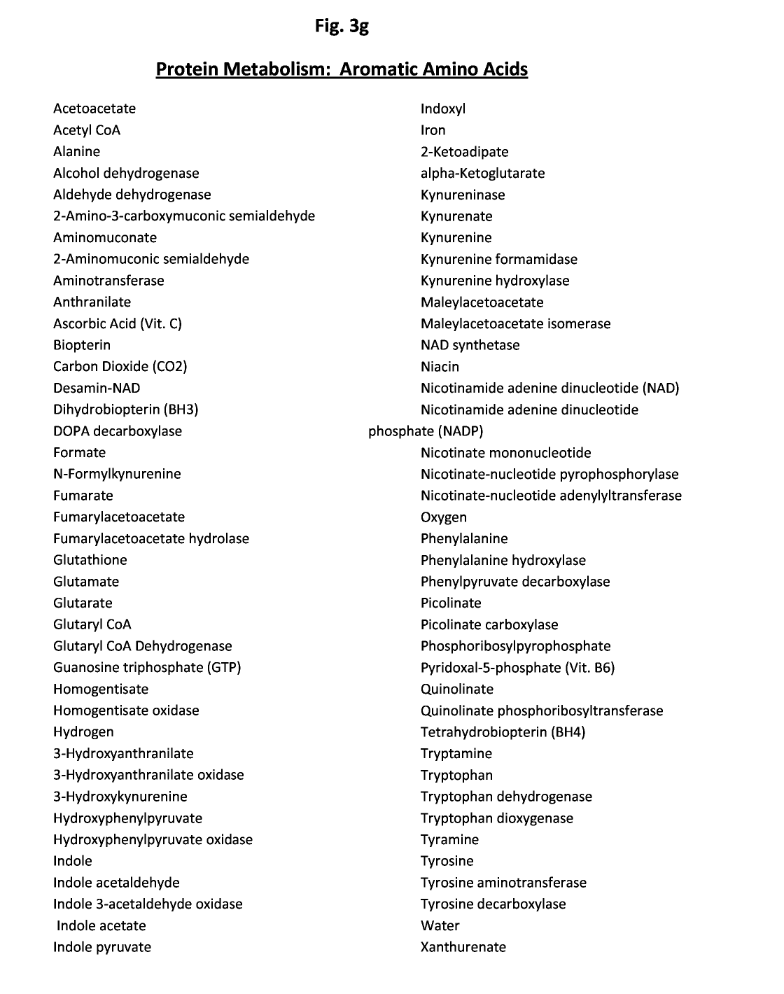

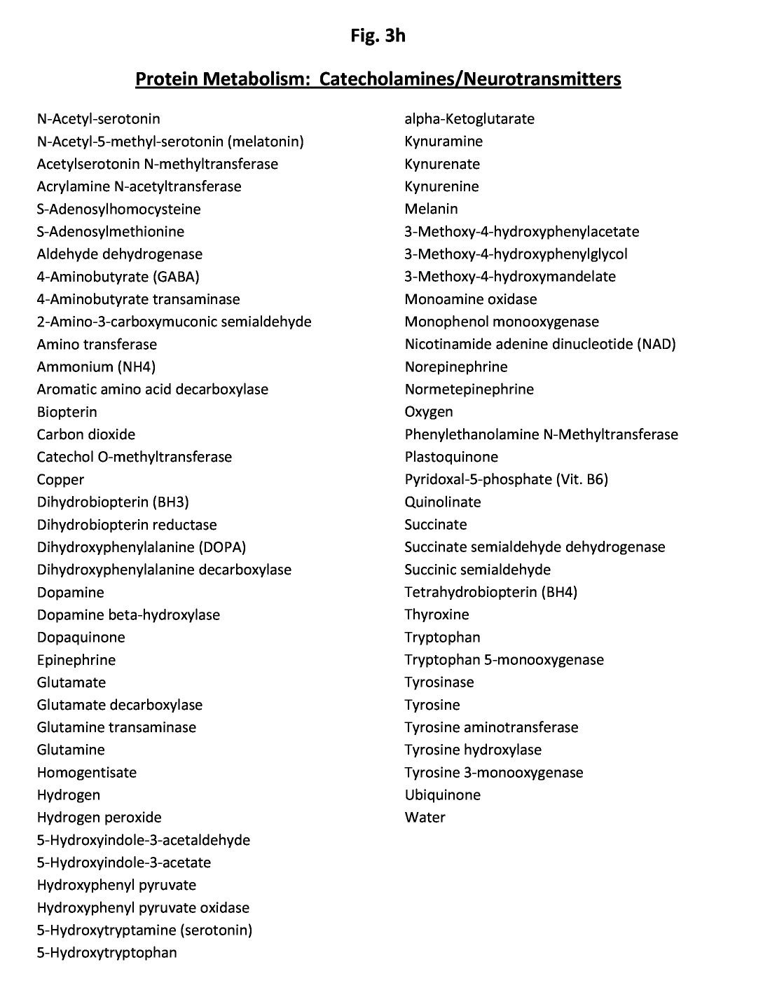

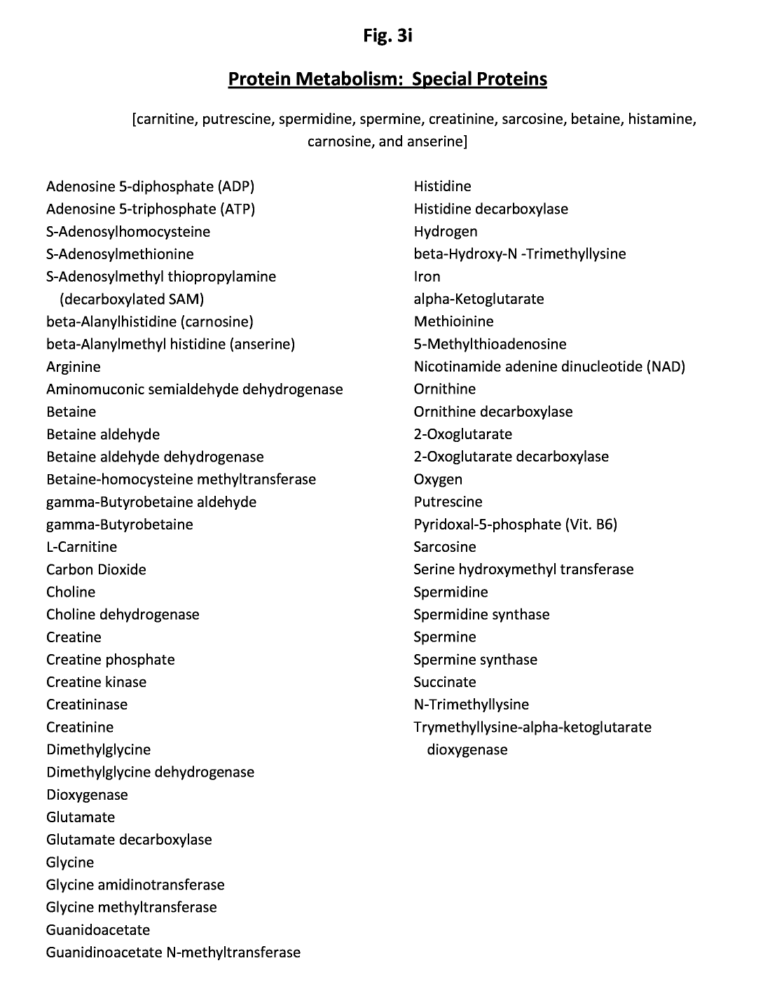

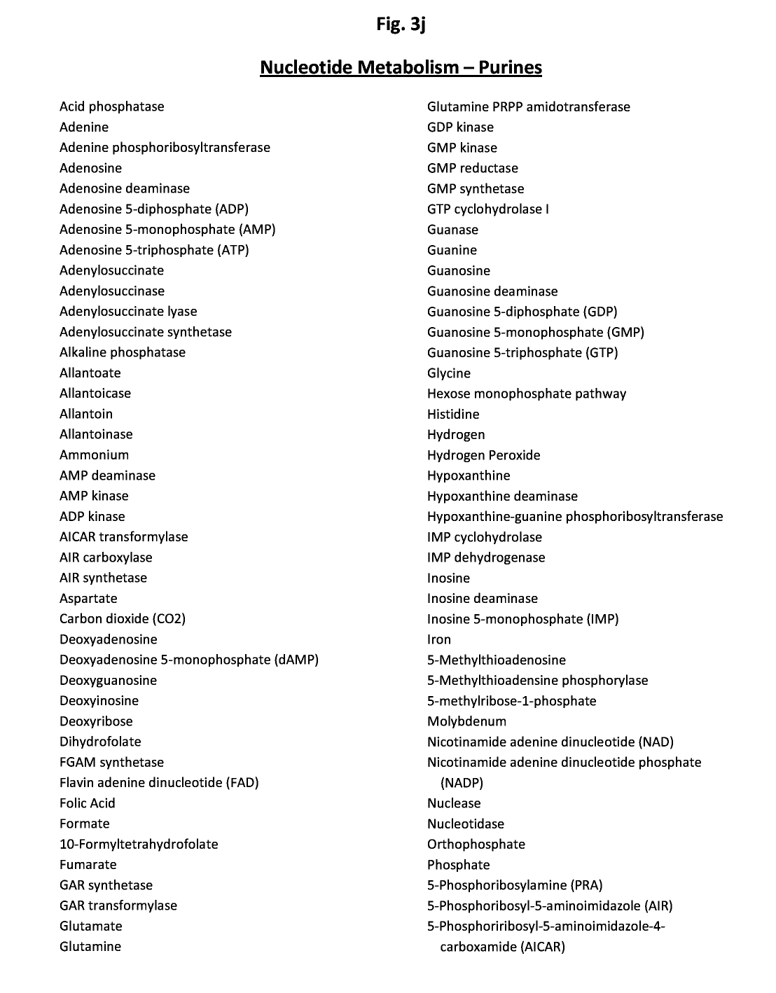

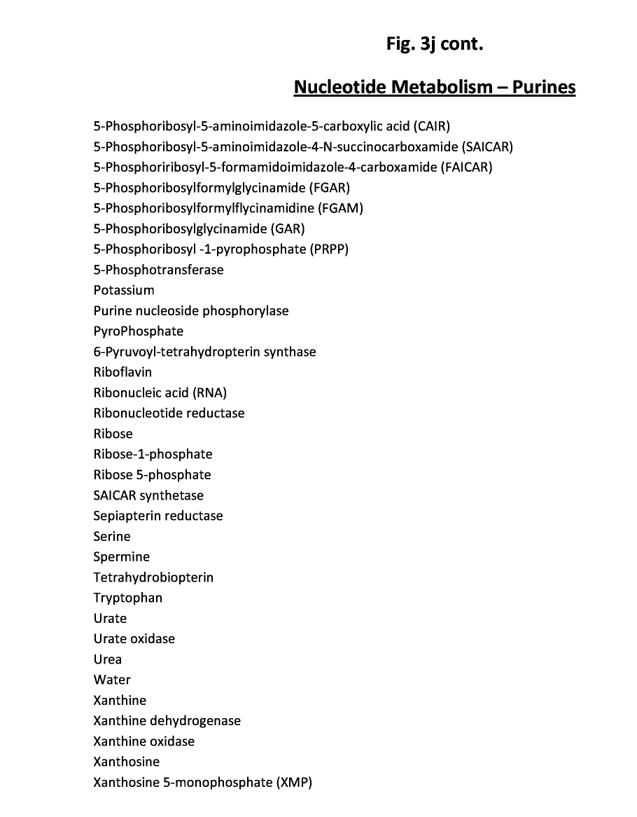

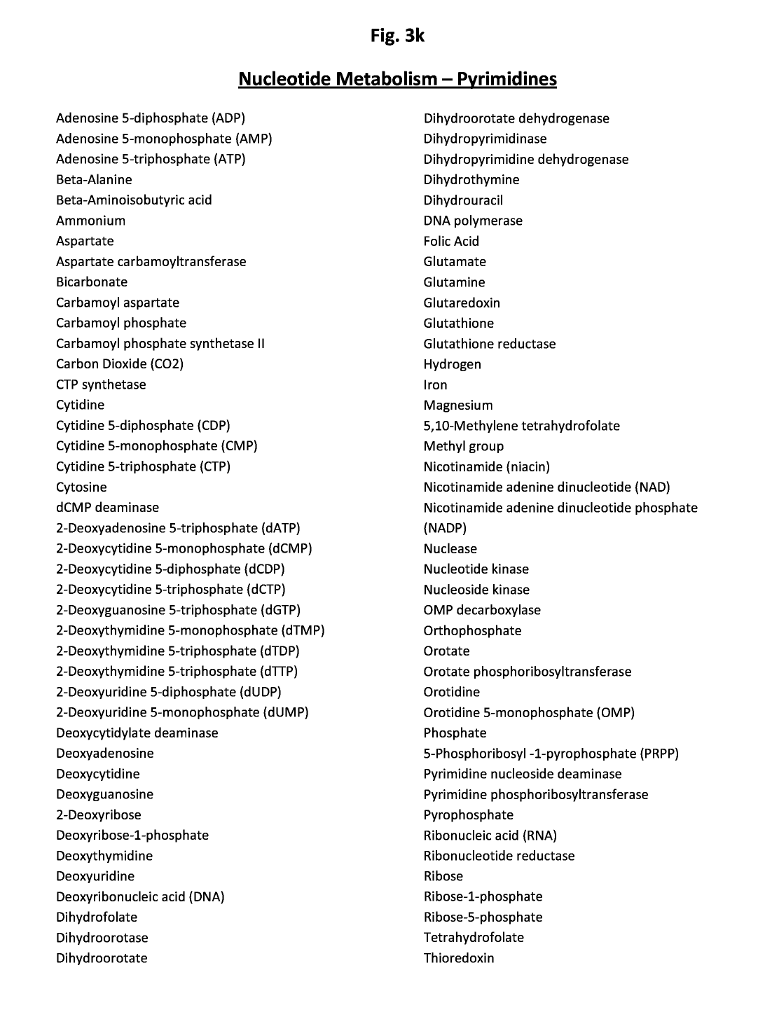

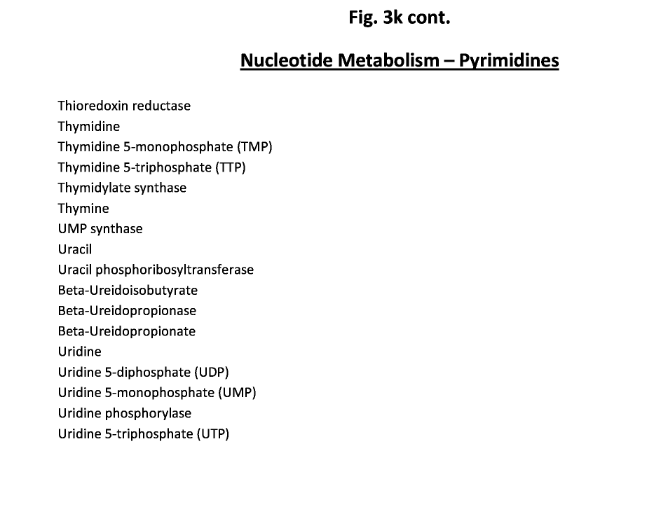

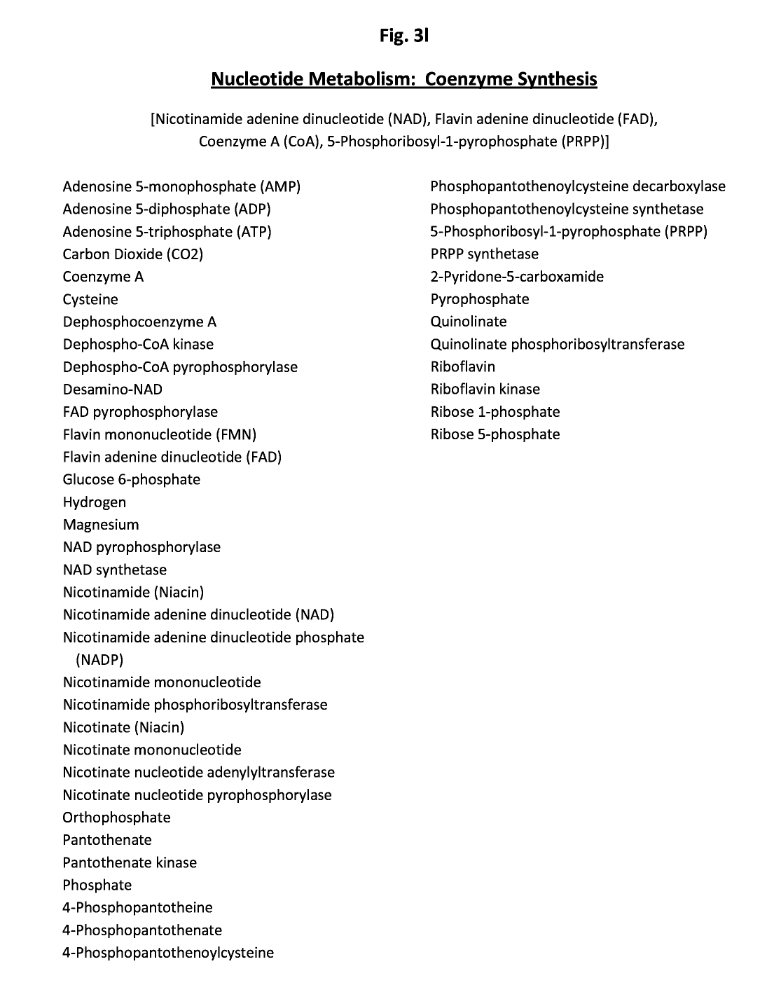

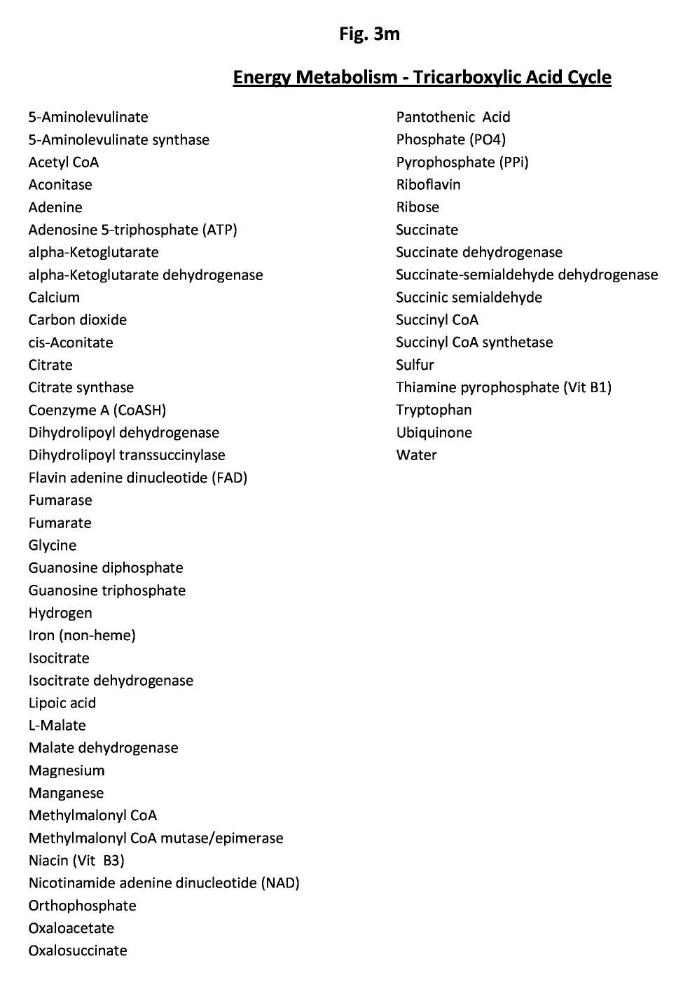

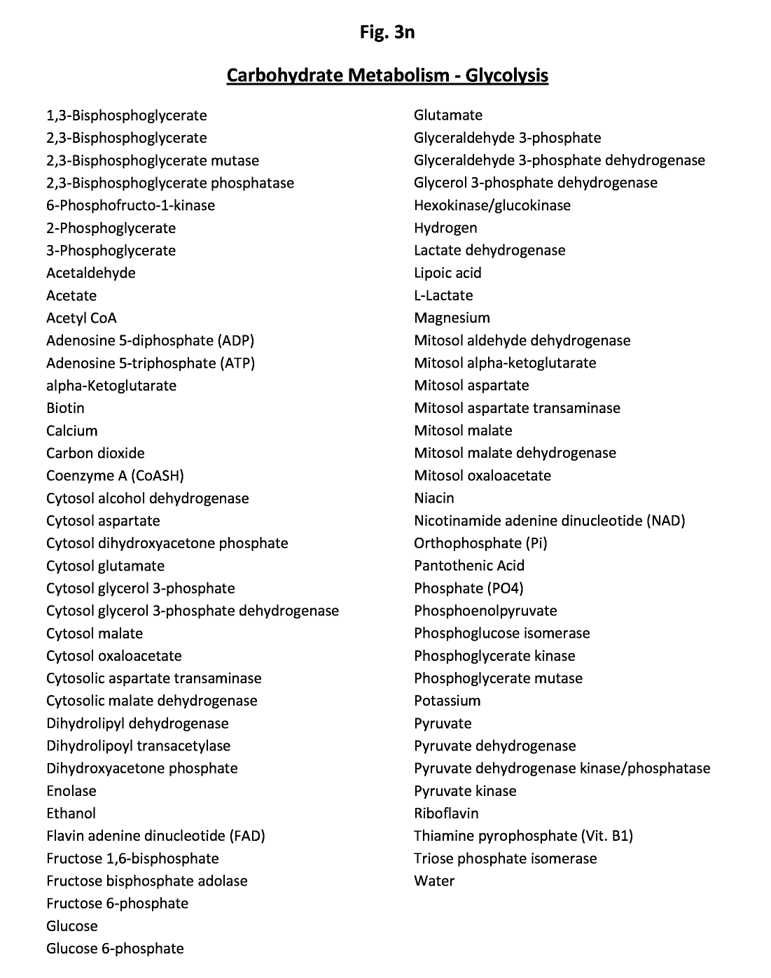

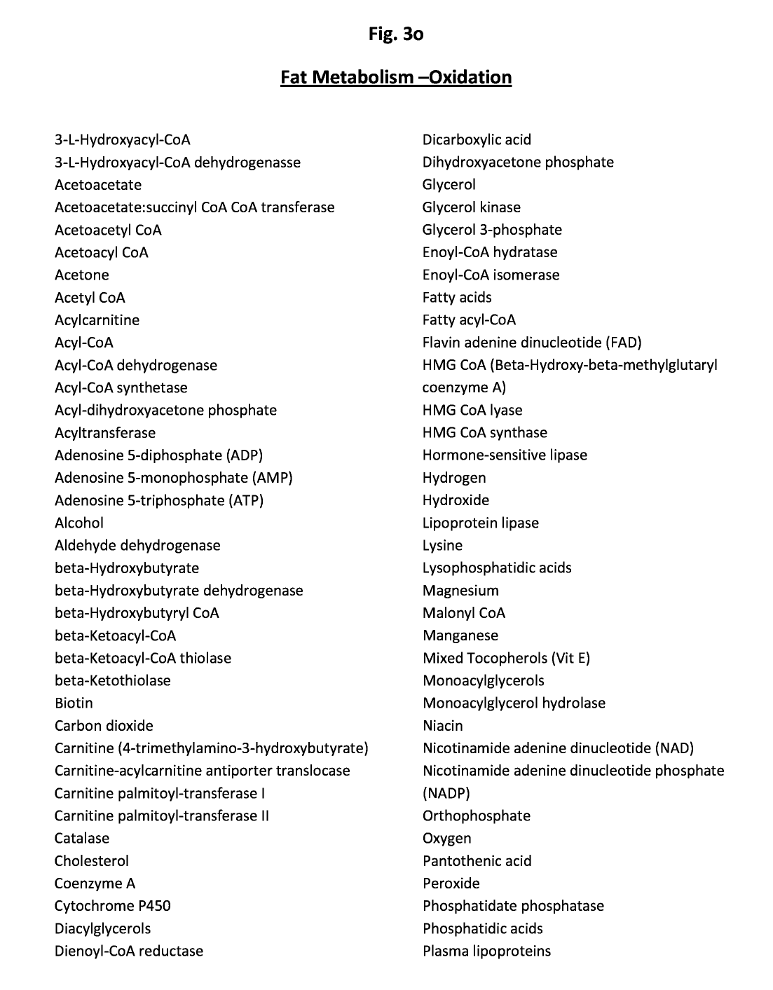

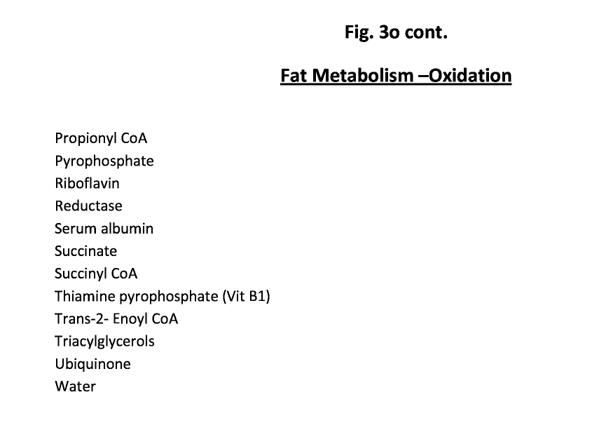

After analyzing the data, the computer software displays the data in a graphical format, ranking the body's response from high to low. Current applications of biocommunication or bioenergetics primarily focus upon determining which organ systems or meridians are imbalanced and/or which medicinal or nutritional products will be the most effective. The unique set of libraries in the present invention consist of comprehensive lists of components in all of the biochemical pathways within the body, see FIGS. 3a-3g, which provide a way for biocommunication technology to assess imbalances at a more basic, biochemical level. This new evaluation tool can provide a healthcare practitioner with invaluable information regarding an individual's ability to metabolize protein, carbohydrates, fat, energy, nucleotides and hormones which is fundamental to the proper functioning of the body.

Most chronic health disorders are the result of multiple poorly regulated or obstructed metabolic pathways. When the body's metabolic pathways are strained at multiple points, they are incapable of utilizing the full spectrum of nutrients available to them, either through the diet or through supplementation. Therefore, it is crucial to address fundamental metabolic imbalances as soon as possible in a patient's treatment program. With this in mind, the use of the present invention with a biocommunication or bioenergetic device provides a precise manner in which therapeutic regimens may be targeted at specific biochemical imbalances in order to achieve the best clinical results. Many nutrients serve as enzyme cofactors or are otherwise instrumental in the proper functioning of the body's metabolic pathways. A particular nutrient may perform a crucial role in more than one metabolic pathway. For example, the trace mineral, manganese, is essential for the activity of several enzymes, most importantly, arginase, pyruvate carboxylase, phosphotransferase and lipoprotein lipase affecting protein, carbohydrate, energy and fat metabolism respectively. Therefore, a deficiency of manganese is likely to cause imbalances in all four metabolic functions resulting in a myriad of seemingly unrelated symptoms making it nearly impossible for a practitioner that relies on symptomatology to accurately diagnose or treat the underlying metabolic problem. The use of the invention with a biocommunication or bioenergetic device can help pinpoint the sources of biochemical imbalances as well as reveal any commonality in causes allowing the practitioner to develop a treatment regimen targeted at correcting the underlying metabolic problem rather than simply attempting to alleviate the symptoms, see paragraph 28.

The goal of any treatment regimen is to remove the sources of stress and bring the individual's body back into balance. The inclusion of the libraries in the invention in any biocommunication or bioenergetic protocol is capable of giving the practitioner the type of information needed to develop treatment regimens that adequately support and balance impaired metabolic pathways without causing stress on either upstream or downstream pathways. Clinical research has found the implementation of the invention in various biocommunication or bioenergetic protocols has increased the healthcare practitioner's effectiveness at developing treatment programs that resolve difficult health problems compared to other protocols that either treat only one or two stressors with the greatest deviation from normal, treat stressors in a particular order, or treat all stressors discovered during the first biocommunication evaluation.

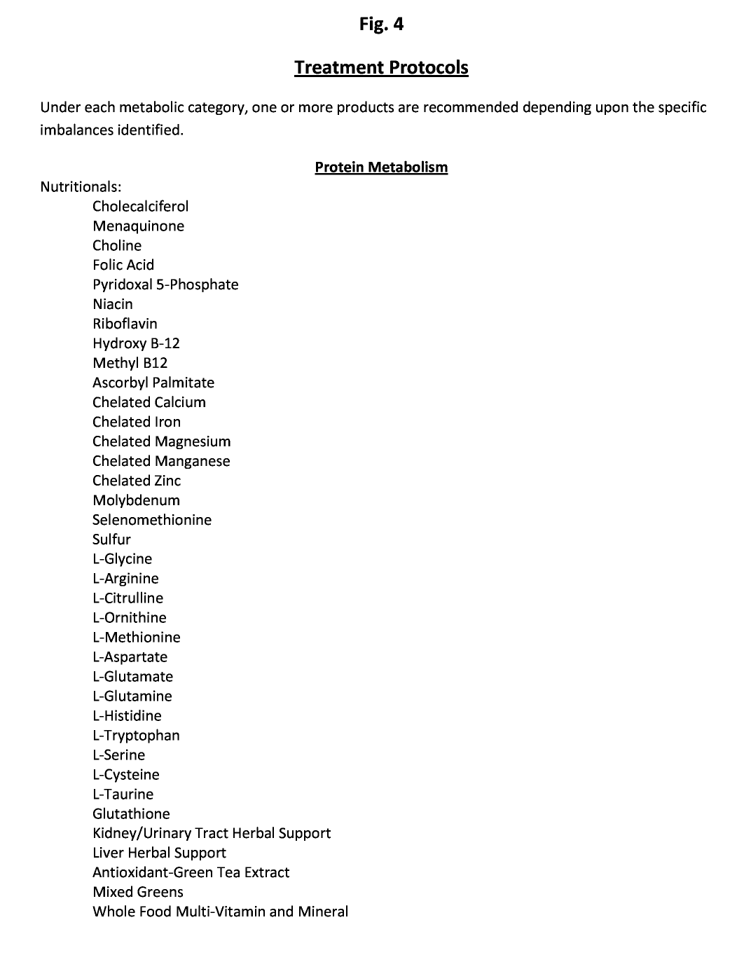

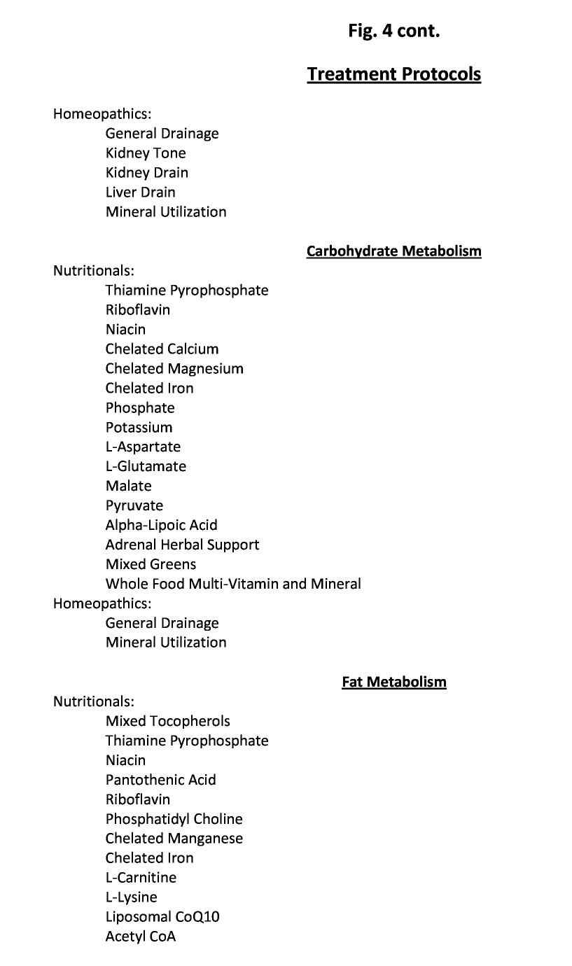

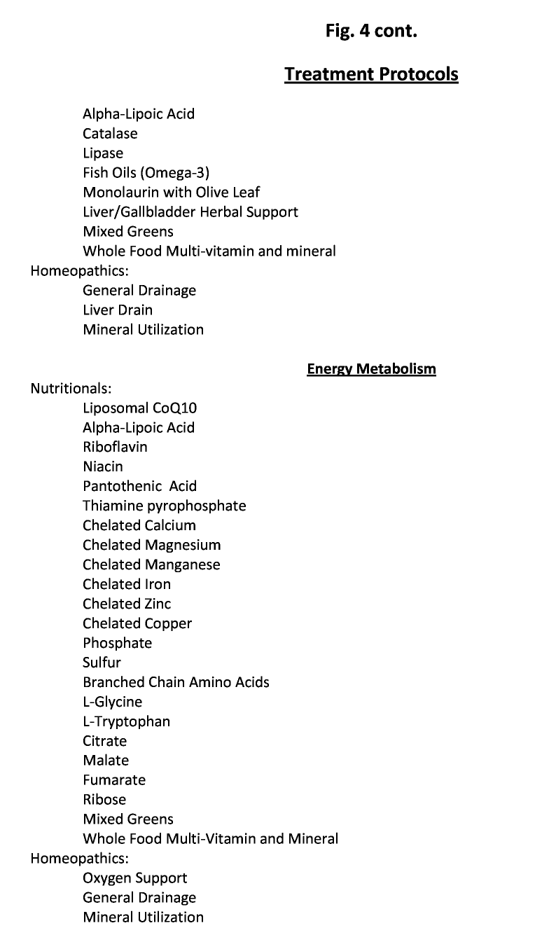

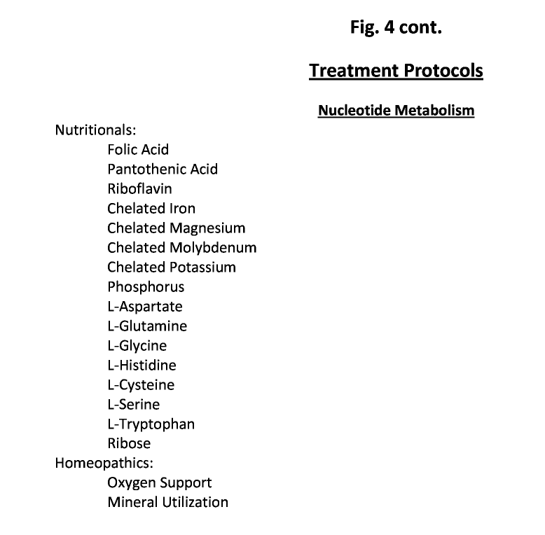

Based on the results of a biocommunication protocol utilizing the invention, an individual is treated with a combination of homeopathic remedies, botanicals, enzymes and nutritional supplements specifically developed to correct the metabolic imbalances identified. There are specific therapeutic products used for each metabolic imbalance, see FIG. 4. As treatment progresses, the specific therapeutic regimen will change as one pathway is balanced and another one is identified for treatment. In the final phase of treatment, very little nutritional support is required. A maintenance program may be necessary to provide continued support for the metabolic pathways in order to counter an individual's inherited weaknesses and prevent progression of simple metabolic imbalances towards symptoms and disease.

Compared to other approaches to healthcare, the invention is simpler to implement thereby reducing stress on the individual; is more affordable because only treatments that are known to resolve the problem are recommended rather than taking a trial and error approach; and is more therapeutically effective, resulting in a faster rate of symptom mitigation and improvement in overall health. In broader terms, the invention can have a beneficial effect on the economy by improving the population's health through disease prevention thereby reducing absences from the workplace and the need for expensive healthcare programs. The present invention provides a comprehensive health assessment tool that is enabling people who have exhausted all hope due to chronic illnesses caused by impaired metabolism to lead vital and productive lives.

The following is an example of a clinical application, in which the invention was implemented on a Zyto Elite biocommunication device, although it can be used in the same manner on any biocommunication or bioenergetic device currently available on the market. Zyto employs the principles of biology, quantum physics, and the science of information to facilitate meaningful communication between computers and the body. A hand cradle is used in the Zyto system as the interface between the computer and the body. The hand of the individual being tested rests on the hand cradle with the palm of the hand and the five fingers each contacting a separate conductive plate that is connected to the PC board or computer inside the hand cradle. The hand cradle is connected to a computer using a USB port, and through the hand cradle the individual receives a stimulus from the computer. The computer then measures the changes in the electrical properties of the skin of the individual's hand caused by the stimulus, known as a galvanic skin response.

Each stimulus from the computer is referred to as a “VSI”, an acronym for Virtual Stimulus Item. VSIs are created in the software using a proprietary process wherein a unique binary string, sometimes refer to as a “binary signature,” is generated, converted to a signal, and that signal is then linked to the physical item. The physical items include nutritional supplements, drugs, organ systems, allergens, and, in our invention, components of the body's biochemical pathways. The specifics of the linking process are proprietary to Zyto but it has basically been accomplished when VSI data is transmitted in the form of an electromagnetic non-radiating wave to the hand cradle. Once the software indicates the linking process is complete, the VSI becomes a stimulus looking for a response from the body in a scanning sequence called a biosurvey.

The objective of the stimulus-response exercise of the biosurvey is to determine the shift the selected VSI or VSIs create in coherence as measured at the contact points on the hand. The biosurvey sequence involves a base line measurement, the introduction of the VSI, and a response measurement. If the energetic posture of the response, measured by the hand cradle, differs from the baseline then the VSI is assumed to have influenced that shift. The only input from the operator is the selection of the biosurvey or the VSIs to use in the biosurvey.

When a biosurvey is initiated, the computer first queries the hand cradle to see if it is properly connected with the hand resting in it. The computer then samples the digital data coming from the hand. This sampling period lasts for a few seconds, allowing the hand to acclimate to the hand cradle. Next, the hand cradle will take a baseline sampling, measuring electrical resistance or conductance of the skin at all contact points. Subtle changes in electrical resistance at each point are then analyzed using a proprietary algorithm to determine a coherent state. This data is then transmitted from the hand cradle to the computer. The computer then sends a VSI signal to the hand cradle. This signal creates a rapid burst of data, sometimes referred to as a “ping.” The hand cradle then takes a response reading measuring electrical characteristics of the skin at all contact points and transfers the data to the computer.

The computer then analyzes the baseline against the response and correlates it with the VSI signal to determine coherence. The variance between the baseline and the response is expressed numerically as a deviation ratio. If the coherence of the response is more coherent than the baseline the deviation ratio will be positive; if the response is less coherent the deviation ratio will be negative. If the coherence of the response is the same as the baseline the deviation ratio will be zero. Additionally, the computer will determine the degree of variance in coherence and will assign a number, the greater the number the more or less coherent the response. The responses to the items in the biosurvey will be ranked according to their deviation ratios and shown in the final report. The healthcare practitioner then takes this data into account when making his therapeutic choice, giving greater consideration to items for which the individual shows a large shift in coherence or a greater deviation ratio.

In this example of a clinical application of the invention, the comprehensive libraries in FIGS. 3a-3g were programmed into the Zyto software becoming VSIs that were used in the biosurveys. To begin the evaluation, the baseline coherence state of the client was established. Then the VSIs were grouped and transmitted to the client through the hand cradle, a process referred to as scanning. The first scan consisted of VSIs of all of the items separated into groups under the six main categories in FIG. 2. The client showed significant negative coherence for protein metabolism, energy metabolism, carbohydrate metabolism, fat metabolism with deviation ratios of −520, −382, −401 and −282, respectively. Nucleotide metabolism and hormone metabolism showed small deviations ratios of 32 and −18, respectively.

Beginning with protein metabolism, the main category with the highest deviation ratio, VSI's were scanned according to the sub-categories listed in FIG. 2. The sub-category showing the largest deviation ratio (−622) in the protein metabolism category was urea cycle. The VSI for each item in this sub-category was then scanned. Several VSIs showed large deviation ratios with manganese and arginase being the highest at −581 and −329, respectively. The same scanning procedure was followed for the sub-categories of the other four main categories with significant deviation ratios. Glycolysis, tricarboxylic acid cycle and fatty acid oxidation yielded the highest deviation ratios from their respective sub-categories. Again, the VSI for each item in each of these sub-categories was scanned. Manganese and an enzyme requiring the mineral for activation (pyruvate carboxylase in glycolysis, phosphotransferase in tricarboxylic acid cycle and lipoprotein lipase in fatty acid oxidation) appeared in the list of top five VSI's with the largest deviation ratios in their respective sub-categories.

The top five VSIs with the largest deviation ratios from each of the sub-categories with significant deviation ratios were scanned together to narrow down the treatment options. Manganese showed the highest deviation ratio of 662. The healthcare practitioner recommended a nutritional supplement containing 5 mg. of manganese to be taken once per day. In four weeks, the client was re-tested and neither manganese nor any of the enzymes requiring manganese as a co-factor appeared on the list of VSIs with the largest deviation ratios. As specified in the invention, the programming of comprehensive libraries of biochemical pathways into a biocommunication device for use in biosurveys allowed the healthcare practitioner to pinpoint the underlying cause of certain metabolic imbalances and to develop an effective therapeutic regimen accordingly.

Having thus described the invention, what is claimed as new and desired to be secured by Letters Patent is:

1. A method of bioenergetic health assessment comprising:
a transmitting a first electrical signal through or across the surface of a subject's epidermis,
measuring said first electric signal to establish a baseline signal,
transmitting a second electrical signal through or across said epidermis, said second electrical signal comprising a signature of a virtual stimulus item,
measuring said second electrical signal,
comparing said measurement of said second electrical signal to said measurement of said baseline signal to determine signal coherence.

2. The method as in claim 1, further comprising the step of assessing deviation from coherence to identify a physiological imbalance associated with said virtual stimulus item.
